(19) United States
(12) Patent Application Publication
(10) Pub. No.: US 2011/0030071 A1
(43) Pub. Date: Feb. 3, 2011

(54) Transgenic Animal Model of Cancer and Methods of Use

(76) Inventors: Ervin H. Epstein, JR., Oakland, CA (US); Po-Lin So, Oakland, CA (US); Jean Tang, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/811,066

(22) PCT Filed: Apr. 9, 2009
(86) PCT No.: PCT/US09/40072

§ 371 Date: Sep. 22, 2010

Related U.S. Application Data
(60) Provisional application No. 61/044,428, filed on Apr. 11, 2008.
(60) Provisional application No. 61/044,821, filed on Apr. 14, 2008.

(51) Int. Cl. G01N 33/15; A01K 67/027

(52) U.S. Cl. 800/3; 800/10

(57) ABSTRACT

The invention features a transgenic mouse model of cancer, exemplified by basal cell carcinoma, which finds use in identification of anti-cancer drugs. The transgenic mouse is characterized by a heterozygous defect in a Patch gene (Ptch^{+/-}) and an inducible suppression of functional p53.

### CROSS-REFERENCE TO RELATED APPLICATION

This application claims priority benefit of U.S. provisional application Ser. No. 61/044,428, filed Apr. 11, 2008 and U.S. provisional application Ser. No. 61/044,821, filed Apr. 14, 2008, which applications are incorporated herein by reference in their entirety.

### BACKGROUND

Skin cancers represent one of the most common cancers, with more than 1 million cases in America alone on an annual basis. Three types of skin cancer are the most prevalent: basal cell carcinoma (BCC), squamous cell carcinoma (SCC), and melanoma.

The Hedgehog (HH) family of intercellular signaling proteins has some essential functions in patterning both invertebrate and vertebrate embryos. Identified as an important regulator of segment polarity and tissue organization in flies, the HH pathway has been shown to play a significant role in cutaneous carcinogenesis in humans. The Sonic hedgehog (SHH) pathway is implicated in the etiology of the most common human cancer, the basal cell carcinoma (BCC). Mutations in the receptor of SHH, the patched gene (Ptch1), have been characterized in basal cell carcinomas (BCCs) of the skin, in squamous cell carcinomas (SCCs), and in the rare genetic syndrome nevoid BCC. (see, e.g., Lupi, Int J. Dermatol. 2007 November; 46(11):1113-7; Asplund et al. Br J. Dermatol. 2005 May; 152(5):868-73).

Mice having a heterozygous defect in Ptch1 (Ptch1+1−) (Goodrich et al, Science, 277 (5329): 1109-13, 1997) develop primordial follicular neoplasms resembling human trichoblastomas. Exposure to ultraviolet radiation or ionizing radiation results in an increase in the number and size of these tumors in these mice, as well as a shift in their histologic features so that they more closely resemble human BCC (Aszterbaum et al. Nat. Med. 1999 November; 5(11):1285-91; US 2003/0186309; U.S. Pat. No. 6,946,257; U.S. Pat. No. 6,551,782; U.S. Pat. No. 6,429,354). The mouse BCC and trichoblastoma-like tumors resemble human BCC in their loss of normal hemidesmosomal components, presence of p53 mutations, frequent loss of the normal remaining Ptch allele, and activation of hedgehog target gene transcription.

### SUMMARY

The invention features a transgenic mouse model of cancer, exemplified by basal cell carcinoma, which finds use in identification of anti-cancer drugs. The transgenic mouse is characterized by a heterozygous defect in a Patch1 gene (Ptch1^{+/-}) and an inducible suppression of functional p53.

Accordingly, the present disclosure provides a method for screening anti-cancer agents comprising administering a candidate agent to a transgenic mouse having a heterozygous mutation in a Patched1 gene and having keratinocytes characterized by inducible suppression of functional p53 gene expression, wherein suppression of p53 gene expression is induced either prior to, with or after administration of said candidate agent, wherein the presence or absence of an effect upon the cancer phenotype in said mouse is indicative of the anti-cancer activity of the candidate agent.

In related embodiments, the transgenic mouse is at least heterozygous for a floxed p53 gene and comprises at least one copy of a transgenic construct comprising a keratinocyte-specific promoter operably linked to a sequence encoding a fusion protein comprising Cre and at least a ligand-binding domain of an estrogen receptor (ER), wherein suppression of functional p53 gene expression in keratinocytes is induced by administration of a ligand for the ER ligand binding domain. In further related embodiments, the cancer is basal cell carcinoma. In further embodiments the transgenic mouse develops visible basal cell carcinoma within about 3 months or about 5 months of induction of suppression of functional p53 gene expression. In other embodiments, the mouse is optionally irradiated prior to, with or after administration of said candidate agent. In related embodiments, the mouse is optionally irradiated prior to, with or after administration of said candidate agent, and can be irradiated at the age of about 8 weeks. In further related embodiments, the transgenic mouse develops visible basal cell carcinoma within about 3 months or about 5 months of induction of suppression of functional p53 gene expression.

The present disclosure also provides a transgenic mouse comprising a heterozygous mutation in a Patched1 gene and having keratinocytes characterized by inducible suppression of functional p53 gene expression, wherein suppression of p53 gene, wherein following suppression of functional p53 gene expression, the mouse develops tumors presents with basal cell carcinoma.

In an exemplary embodiment, the transgenic mouse is at least heterozygous for a floxed p53 gene and comprises at least one copy of a transgene construct comprising a keratinocyte-specific promoter operably linked to a sequence encoding a fusion protein comprising Cre and at least a ligand-binding domain of an estrogen receptor (ER), wherein suppression of functional p53 gene expression in keratinocytes is induced by administration of a ligand for the ER ligand binding domain. In related embodiments, the transgenic mouse is homozygous for the floxed p53 gene. In further embodiments, the transgenic mouse develops visible basal cell carcinoma within about 3 months or about 5 months of induction of suppression of functional p53 gene expression.

Other features of the invention and its related disclosure are provided below, and will be readily apparent to the ordinarily skilled artisan upon reading the present disclosure.

### BRIEF DESCRIPTION OF THE DRAWINGS

The invention is best understood from the following detailed description when read in conjunction with the accompanying drawings. Included in the drawings are the following figures:

FIG. 1 provides the timeline followed for the administration of tamoxifen and/or irradiation. In Experiment (Exp) 1, mice were injected Tam at the age of 6 weeks, followed by exposure to 5 Gy of ionizing radiation (IR). In Exp 2, mice were irradiated at the age of 8 weeks before being injected with Tam at the age of 9 weeks. In Exp3, mice were injected with Tam at the age of 6 weeks but were not irradiated.

FIG. 2 shows mice that were administered tamoxifen at the age of 6 weeks and were irradiated at the age of 8 weeks. Panels A-D show the mice of the genotype Ptch1^{+/-}; p53^{fl/fl}. Panels E-H show mice of the genotype Ptch1^{+/-}; K14-Cre-ER; p53^{fl/fl}. Mice that are heterozygous for Ptch1 mutation and lack p53 expression in keratinocytes (E-H) show an acceleration of BCC carcinogenesis compared to Ptch1^{+/-}; p53^{fl/fl} mice (A-D).

FIG. 3 shows mice of the genotype Ptch1^{+/-}; K14-Cre-ER; p53 Panels A-D show the mice that were injected with Tam at the age of 6 weeks and irradiated at 8 weeks of age (Exp1). Panels E-H show the mice that were irradiated at the age of 8 weeks and injected with Tam at the age of 9 weeks (Exp2).

FIG. 4 shows mice of the genotype Ptch1^{+/-}; K14-Cre-ER; p53^{fl/fl} that were injected with tamoxifen (Tam) at the age of 6 weeks but were not irradiated (left panel) and histology of biopsied tumor from these mice (right panel).

FIG. 5 depicts mice of the genotype Ptch1^{+/+}; K14-Cre-ER; p53^{fl/fl} (left panel) and histology of biopsied tumor from these mice (right panel). Deletion of p53 by injecting Tam at the age of 6 weeks followed by irradiation at the age of 8 weeks causes the development of SCC in these mice.

FIG. 6 illustrates the percent of mice of the genotype Ptch1^{+/-}; K14-Cre-ER; p53^{fl/fl}; genotype Ptch1^{+/-}; p53^{fl/fl} that were free of BCC when injected with Tam at the age of 6 weeks and irradiated at the age of 8 weeks. Ptch1^{+/-}; p53^{fl/fl} mice that were not injected with Tam served as an additional control group. Percent of mice of the genotype Ptch1^{+/+}; K14-Cre-ER; p53^{fl/fl} that were free of SCC when injected with Tam at the age of 6 weeks and irradiated at the age of 8 weeks are also depicted.

FIG. 7 presents a comparison between mice of the genotype Ptch1^{+/-}; K14-Cre-ER; p53^{fl/fl} that were injected with Tam at the age of 6 weeks and then irradiated at 8 weeks and mice of the genotype Ptch1^{+/-}; K14-Cre-ER; p53^{fl/fl} that were irradiated at 8 weeks and then injected with Tam at the age of 9 weeks.

FIG. 8 shows histology of BCC from mice of the genotypes indicated.

### DETAILED DESCRIPTION OF EMBODIMENTS OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a candidate agent” includes a plurality of such agents and reference to “the gene” includes reference to one or more genes and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “transgene” is used herein to describe genetic material which has been or is about to be artificially inserted into the genome of a mammal, particularly a mammalian cell of a living animal.

By “transgenic animal” is meant a non-human animal, usually a mammal, having a non-endogenous (i.e., heterologous or foreign) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art. A “transgene” is meant to refer to such heterologous nucleic acid, e.g., heterologous nucleic acid in the form of an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). Accordingly, when a DNA molecule is artificially introduced into the cells of an animal, a “transgenic animal” is produced. The DNA molecule is called a “transgene” and may contain one or many genes. By inserting a transgene into a fertilized oocyte or cells from the early embryo, the resulting transgenic animal may be able to transmit the foreign DNA stably in its germline.

A “knock-out” of a gene means an alteration in the sequence of the gene that results in a decrease of function of the target gene, and includes decrease in function such that target gene expression is undetectable or insignificant. Transgenic knock-out animals can be characterized as comprising a heterozygous knock-out of a target gene (i.e., with a knockout of a first allele and a second allele that is functional (e.g., wild-type)) or a homozygous knock-out of a target gene. “Knock-outs” as used herein also include conditional knock-outs (also referred to as “inducible” knock-outs), where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally. In the present disclosure, the transgenic mice are characterized as comprising a heterozygous knock-out of a Patch1 gene, and an inducible knock-out of a p53 gene, where the transgenic animal can be heterozygous or homozygous, usually homozygous, for a construct that provides for the inducible p53 knock-out in a target tissue, e.g., skin, e.g., keratinocytes.

“Homozygous” state means a genetic condition existing when the same alleles reside at corresponding loci on homologous chromosomes. In contrast, “heterozygous” state means a genetic condition existing when different alleles reside at corresponding loci on homologous chromosomes.

“Site specific recombinases” are enzymes that are present in some viruses and bacteria and have been characterized to have both endonuclease and ligase properties. Site specific recombinases catalyze at least the following four events (a) deletion of a DNA fragment flanked by “compatible site-specific recombinase targeting sites” (SSRTS) in the same orientation (e.g. head-to-tail or tail-to-head); (b) inversion of a DNA fragment flanked by compatible SSRTS in opposite orientation (e.g. head-to-head or tail-to-tail); (c) integration of a cyclic DNA fragment containing a SSRTS into a compatible SSRTS; and (d) chromosomal translocation between compatible SSRTS located on different chromosomes. To perform those reactions, the site-specific recombinase has typically at least the following four activities: (1) recognition of one or two specific DNA sequences; (2) cleavage of said DNA sequence or sequences; (3) DNA topoisomerase activity involved in strand exchange; and (4) DNA ligase activity to reseal the cleaved strands of DNA (Sauer, B., Cur. Opin. Biotech. 5: 521-527, 1994).

The term “recombinase” or “site-specific recombinase” refers to enzyme(s) that carry out site specific recombination (SSR) to alter the DNA structure. This definition includes transposases, lambda integration/excision enzymes, and site-specific recombinases. Well-known examples of recombinases can be found in Cre-lox, FLP/FRT, R/RS, Gin/gix, a pSR1 system, a cer system, and a fim system (e.g. N. L. Craig, Annu. Rev. Genet. 22:17, 1988); Odell et al., Homologous Recomb. Gene Silencing Plants, 1994, pp. 219-270, Paszkowski, Jerzy, ed. Kluwer: Dordrecht, Germany). Additionally, SSR systems have been identified in microorganisms such as phage, bacterium (e.g., E. coli), yeast and the like. This includes the E. coli lambda att P system for integration and excision (Zubko et al. Nature Biotechnology 18:442, 2000) and the Streptomyces phage C31 integrase (Groth et al. Proc. Natl. Acad. Sci. USA 97:5995, 2000). When the SSR system from these microorganisms is introduced into organisms (including plants) different from the organism from which this system had been derived, it behaves in the same way as in the original organism.

“Recombinase site” or “site-specific recombination sequence” means a DNA sequence that a recombinase will recognize in facilitating a recombination event. It will be appreciated that this may be a wild type or mutant recombinase site, as long as functionality is maintained and the recombinase enzyme may still recognize the site, bind to the DNA sequence, and catalyze the recombination between two adjacent recombinase sites.

The term “floxed” refers to the flanking of a genetic element, or a portion thereof, with tandem site-specific sequences. The site-specific sequences may be oriented in the same orientation, i.e., directly repeated (such that the DNA element is removed upon SSR), or in opposite orientations from one another (such that the DNA element is inverted upon SSR). The floxed element may be a single promoter, a transcriptional ‘STOP’ blocking element, a promoter operably linked to a target sequence, or a combination of these and other genetic elements.

A “site-specific recombination substrate” or “SSR substrate” refers to any DNA that is a substrate of SSR, resulting from the action of the site-specific recombinase on recombinase sites. It includes floxed DNA elements, i.e., those DNA elements flanked by recombinase sites.

“Operably-linked” refers to the arrangement of nucleic acid sequences of a nucleic acid so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

“Treating” or “treatment” of a condition or disease includes providing a clinical benefit to a subject, and includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

“Suppression of functional p53 gene expression” as used herein means reduction in production of functional p53 protein in a cell due to, for example, disruption of at least one p53 allele.

“Tissue-specific expression” or “cell type-specific expression” (as in “keratinocyte-specific expression”) as used herein is meant to indicate that expression of a gene is provided predominantly in a target tissue or cell of interest, although expression at lower levels may be observed in non-target tissues or non-target cells.

Transgenic Mouse Model of Cancer Associated with Patch Mutation and Inducible P53 Mutation

The transgenic mouse of the present disclosure is generally characterized as having a heterozygous mutation in a Patched1 gene and having keratinocytes characterized by inducible suppression of functional p53 gene expression, wherein following suppression of functional p53 gene expression in keratinoctyes, the mouse develops basal cell carcinoma. In some examples, the transgenic mouse is at least heterozygous for a floxed p53 gene and comprises at least one copy of a transgene construct comprising a keratinocyte-specific promoter operably linked to a sequence encoding fusion protein of a Cre and at least a ligand-binding domain of an estrogen receptor (ER), wherein suppression of functional p53 gene expression in keratinocytes is induced by administration of a ligand for the ER ligand binding domain. In certain embodiments, the mouse is homozygous for the floxed p53 gene.

The various aspects of the transgenic mouse model will be described in more detail below.

Patch1 Mutant

Patched1 (ptch1) gene encodes a receptor for Sonic hedgehog (Shh), a secreted molecule implicated in the formation of embryonic structures and in tumorigenesis. Ptch1 functions as a tumor suppressor. The transgenic mice of the present disclosure are heterozygous for a Ptch1 mutation, such that one allele of the Ptch1 gene is defective and does not produce a functional Patch1 protein. Mice heterozygous for a Ptch1 mutation are indicated as having the genotype ptch1+/−. Thus ptch1+/−mice carry one functional patch1 allele (represented by ptch1+, and sometimes referred to herein as the “wildtype”) and one mutant allele (represented by ptch1−) of the ptch1 gene. Thus in the context of describing the genotype of the transgenic mice herein, “ptch1” as used herein refers to the ptch1 gene, modifications thereof, mRNA, splice variants thereof, gene with the wild type sequence as well as with mutations. “Mutant ptch1” as used herein refers to a ptch1 gene that does not produce a functional ptch1 gene product, e.g., due to insertion, deletion, substitutions, etc. relative to a functional ptch1 gene.

Exemplary mice lacking a functional copy of the Ptch1 gene useful in production of the transgenic mice of the present disclosure include those described in Goodrich et al, Science, 277 (5329): 1109-13, 1997. In these mice, a portion of exon 1, including the start codon and all of exon 2 of the Ptch1 gene was replaced with lacZ gene by homologous recombination. Protein made from any alternative start codon would lack the first proposed transmembrane domain, flipping the orientation of the protein in the membrane. Thus the ptch1 gene in these mice was knocked out (mutated). Methods for producing mice heterozygous for a Ptch1 mutation are well known in the art.

Inducible p53 Suppression

The transgenic mice of the present disclosure are further characterized as having keratinocytes having inducible suppression of functional p53 gene expression. This is generally accomplished by providing for a conditional knock-out of at least one p53 allele.

Transgenic mice of the present disclosure are at least heterozygous, and optionally homozygous, for a modified p53 gene flanked by site-specific recombinase targeting sites (SSRTS). The mice are also transgenic for at least one copy of a construct encoding a compatible site-specific recombinase that is under control of a promoter functional in keratinocytes, particularly a keratinocyte-specific promoter. Activity of the site-specific recombinase in the keratinocytes is inducible, e.g., by providing that the site-specific recombinase is active upon administration of an inducer agent, as described below in more detail. The recombinase catalyzes site-specific recombination at the modified p53 gene, thereby disrupting production of p53 at that allele, and providing for suppression of functional p53 gene expression in keratinocytes.

Providing a site-specific recombinase having inducible activity can be accomplished in a variety of ways. For example, the mouse can be transgenic for a construct encoding a site specific recombinase expressed as a fusion protein which provides for inducible activity in the presence of an inducing agent. For example, the site specific recombinase can be provided as a fusion protein with at least a ligand binding portion of a hormone receptor (e.g., estrogen receptor). Binding of ligand to the fusion protein results in the translocation of the fusion protein to the nucleus, thus providing access of the recombinase to the target site at the modified p53 allele.

In order to provide for tissue-specific expression in the skin, the site specific recombinase can be expressed under the control of a skin-specific promoter. Exemplary promoters, useful for the generation of the mouse model, include but are not limited to promoters that are active in epidermis, hair follicles of skin, oral epithelia, etc. A number of tissue specific promoters have been isolated and transgenic mouse lines carrying such promoters are widely available. The keratin promoters, K14 and K5 have been particularly useful in targeting the expression of transgenes to the mitotically active basal layer of mouse epidermis and to the outer sheath of hair follicle, oral epithelia, and oesophagus (Wang et al., PNAS 94, 219-226, 1997, Vasioukhin et al., PNAS 96, 8551-8556, 1999). The K10 and involucrin promoters have enabled targeting of gene expression to the differentiating suprabasal layers of epidermis (Bailleul et al., Cell, 62, 697-708, 1990), and a segment of the K1 promoter has been useful in targeting expression to all epidermal layers (Greenhalgh et al., Mol. Carcinog. 7, 99-110, 1993).

In certain embodiments, the site specific recombinase is expressed under the control of an inducible promoter. The tetracycline-inducible system of Gossen and Bujard has been used to regulate inducible expression of several genes (Gossen and Bujard 1992, supra; Furth et al. 1994 Proc. Natl. Acad. Sci. USA 91:9302-9306; Howe et al. 1995 J. Biol. Chem. 270:14168-14174; Resnitzky et al. 1994 Mol. Cell. Biol. 14:1669-1679; Shockett et al. 1995 Proc. Natl. Acad. Sci. USA 92:6522-6526). This system uses a chimeric transcription factor, termed tTA, which is composed of the repressor of Escherichia coli (E. coli) tetracycline-resistance operon (tetR) and the activation domain (carboxyl terminal domain) of virion protein 16 (VP16) of herpes simplex virus (HSV) (Triezebberg et al. 1988 Genes Dev. 2:718-729). The gene of interest is placed downstream of a minimal cytomegalovirus (CMV) 1A promoter, derived from the immediate early CMV genes, which is linked to multiple copies of tetO, the binding site for the tetracycline repressor tetR. In the absence of tetracycline, the tetR portion of the transactivator binds the tetO sequences of the promoter and the VP16 portion facilitates transcription. When tetracycline is present, tetracycline binds the tetR portion of tTA, which in turn prevents binding of the tetR portion to the tetO sequence(s) of the promoter, thus inhibiting transcription. Since even low concentrations of tetracycline are sufficient to block tTA function, and since most mammalian cells can tolerate tetracycline, this system provides a tightly regulated on/off switch for gene expression that can be controlled by varying the tetracycline concentration to which the cells are exposed.

Exemplary p53 conditional-mutant mice were generated by introducing loxP sites in intron 1 and in intron 10 of the p53 gene, i.e., the p53 gene was floxed so that at least a portion of the p53 gene is flanked by loxP sites (Jonkers et al., Nature Genetics 29: 418-425, 2001). Mice homozygous for the floxed p53 gene are described by the genotype p53 fl/fl, while mice heterozygous for the floxed p53 gene are denoted as p53 fl/+, i.e., they carry one wild-type allele and one floxed allele of the p53 gene. Mice of the genotype p53 fl/fl and p53 fl/+ have normal expression levels for the p53 protein. The expression of Cre recombinase in these mice will result in the deletion of the floxed p53 gene. Thus p53 gene is inducibly deleted when Cre recombinase is provided. Cre recombinase is provided by crossing a trangenic mouse carrying a floxed gene to a trangenic mouse carrying the cre gene operably linked to a promoter.

K14 promoter has been particularly useful in directing expression of transgenes (operably linked to it) in mitotically active basal layer of mouse epidermis and to the outer sheath of hair follicle, oral epithelia, and oesophagus (Vassar et al., PNAS 86, 1563-1567, 1999). K14-Cre transgenic embryos recombine genetically introduced loxP sequences selectively in the genomes keratinocytes that reside in embryonic day 14.5 skin, tongue and oesophagus. K14-Cre-ER transgenic mice express Cre recombinase in kertinocytes only when Tamoxifen (Tam) is present (Vasioukhin et al., PNAS 96, 8551-8556, 1999, Wang et al., PNAS 94, 219-226, 1997). ER is a Tam responsive hormone-binding domain of the estrogen receptor. Thus Cre can be expressed specifically in keratinocytes post natally by administering Tam to these mice. Tam can be administered orally, via injection or topically. Topical administration of Tam results in Cre expression in the area of the skin where Tam was applied. Method for generating K14-Cre-ER transgenic mice is described in Vasioukhin et al., supra and in Metzger et al. Methods Mol. Biol., 289: 329-40, 2005. In certain embodiments disclosed herein, Tam is administered via injection. K14-Cre-ER transgenic mice can have a single copy of the K14-Cre-ER transgene, i.e., the transgenic mouse has only one insertion of this transgene in its genome. Alternatively, K14-Cre-ER transgenic mice can have two copies of K14-Cre-ER transgene.

Tamoxifen is an orally active selective estrogen receptor modulator that is used in the treatment of breast cancer and is currently the world's largest selling drug for that purpose. Tamoxifen was discovered by ICI Pharmaceuticals[1] (now AstraZeneca) and is sold under the trade names Nolvadex,® Istubal,® and Valodex.® In addition to Tam, other ligands for estrogen receptor, including but not limited to estrogen, 17.beta.-estradiol and moxestrol, are also useful.

### Methods of Screening for Anti-Cancer Agents

The transgenic mouse of the present disclosure finds particular use in screening candidate agents for anti-cancer activity. By “anti-cancer activity” is meant activity in directly or indirectly mediating any effect in preventing or inhibiting tumor development of growth which may provide for a beneficial effect to the host. “Anti-cancer activity” thus encompasses, for example, prevention of or inhibition of cancer cell and/or tumor development, inhibition of tumor cell growth, reduction of tumor load, and the like. Agents having anti-cancer activity that finds clinical use in treatment of cancer are of particular interest. Of particular interest in the present disclosure is the identification of agents that have an anti-cancer activity that can provide a clinical benefit in treating cancer characterized by a defect in hedgehog signaling, more particularly a defect in Patch1. Of particular interest is the identification of agents having anti-cancer activity in skin cancer, more particularly skin cancer having a Patch1 defect (e.g., a heterozygous or homozygous defect in Patch1), even more particularly basal cell carcinoma.

In general, anti-cancer activity is assessed by evaluating the presence or absence of an effect upon a cancer phenotype in the transgenic mouse, which effect on cancer phenotype is indicative of the anti-cancer activity of the candidate agent. Thus, for example, a reduction in tumor burden in the mouse following administration of a candidate agent indicates the agent has anti-cancer activity.

The screening methods are generally carried out by administering a candidate agent to the transgenic mouse, either prior to, with or, after induction of suppression of functional p53 gene expression. The suppression of functional p53 gene expression can be induced at about 4 weeks, 6 weeks, or 9 weeks prior to or following candidate agent administration. Where the candidate agent and an inducer of functional p53 gene expression are administered together, they may be administered by the same or different routes, or in the same or different compositions.

Optionally, the transgenic mouse can be treated with ionizing radiation prior to, with or after administration of the induction of suppression of functional p53 gene expression in the keratinocytes and/or prior to, with or after administration of said candidate agent. The mouse can be irradiated at about 4 weeks, 6 weeks, 7 weeks or 8 weeks of age prior to or following induction of suppression of functional p53 gene expression.

Any of a variety of candidate agents can be screened for anti-cancer activity. “Candidate agents” is meant to include synthetic, naturally occurring, or recombinantly produced molecules (e.g., small molecule; drugs; peptides; antibodies (including antigen-binding antibody fragments, e.g., to provide for passive immunity); endogenous factors present in eukaryotic or prokaryotic cells (e.g., polypeptides, plant extracts, and the like)); etc.). Of particular interest are screening assays for agents that have a low toxicity for human cells. Candidate agents also include agents that may serve as cancer vaccines, e.g., to provide for production of an immune response in a subject that will in turn have anti-cancer activity.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The candidate agent can be administered in any manner desired and/or appropriate for delivery of the agent in order to examine anti-cancer activity. For example, the candidate agent can be administered topically, by injection (e.g., by injection intravenously, intramuscularly, subcutaneously, and the like), orally, or by any other desirable means. Topical administration is of particular interest.

The screening method can involve administering varying amounts of the candidate agent (from no agent to an amount of agent that approaches an upper limit of the amount that can be delivered successfully to the animal, e.g., within toxicity limits), and may include delivery of the agent in different formulations and routes. The agents can be administered singly or can be combined in combinations of two or more, especially where administration of a combination of agents may result in a synergistic effect.

The ability of a candidate agent to treat a preexisting cancer can be assessed by administering the candidate agent to the transgenic mouse in which suppression of functional p53 gene expression has been induced, and evaluating modulation of a cancer phenotype. Modulation of a cancer phenotype can be assessed by evaluating the present or absence of an effect on, for example, tumor burden, number of tumors, tumor size, metabolic activity of tumor cells, and the like.

The ability of a candidate agent to facilitate prevention of cancer can be assessed by administering the candidate agent prior to induction of the conditional p53 gene defect in the animal and/or prior to exposure of the animal to radiation. “Prevention” of cancer refers to reduction in the incidence and/or severity of tumors in the animal relative to the incidence and/or severity of cancer expected in the absence of intervention (e.g., without administration of an agent having anti-cancer activity).

As noted above, the model disclosed herein can facilitate screening of cancer vaccines. A “vaccine” is an agent that, following administration, facilitates induction of an immune response against a target antigen(s) so that the humoral, cellular, or humoral/cellular immune response elicited can facilitate inhibition of a cancer phenotype. Vaccines include those that provide for prevention (e.g., to prevent development of a cancer) and those that are therapeutic (e.g., suitable for administration to a host having a pre-existing tumor).

In this embodiment, anti-cancer activity can also be assessed by examining the induction of an anti-cancer immune response (e.g., by evaluating humoral and/or cellular responses that are specific for the antigen). The candidate anti-cancer vaccine can be administered prior to induction of the suppressed functional p53 gene expression phenotype (e.g., to evaluate the ability of the vaccine to inhibit tumor development) or can be administered to transgenic mice having pre-existing tumors (i.e., after induction of the suppressed functional p53 gene expression phenotype).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### P53 Deletion in Keratinocytes Accelerates BCC Carcinogenesis In PTCH1^{+/-}Mice

Mice with the K14-Cre-ER transgene (Metzger et al. Methods Mol. Biol., 289: 329-40, 2005) and mice with a floxed p53 allele (Jonkers et al. Nature Genetics, 29: 418-425, 2001) were bred with Ptch1+/−mice (Aszterbaum et al. Nat. Med. 1999 November; 5(11):1285-91) to generate Ptch1+/−K14-Cre-ER p53fl/fl and Ptch1+/−p53fl/fl mice. Ptch1^{+/-}; K14-Cre-ER; p53^{fl/fl} mice are heterozygous for Ptch1 mutation and have p53 gene deleted in the keratinocytes upon administration of an estrogen receptor ligand, such as tamoxifen. Ptch1^{+/-}; p53^{fl/fl} mice, heterozygous for Ptch1 mutation but with wild type p53 gene (flanked by Cre sequences), served as control. Mice were genotyped by PCR analysis of genomic DNA extracted from tail biopsies.

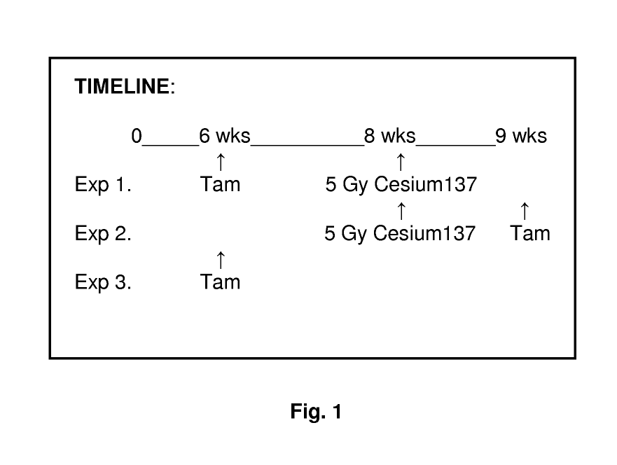

Ptch1^{+/-}; K14-Cre-ER; p53^{fl/fl} mice and Ptch1^{+/-}; p53^{fl/fl} mice were administered tamoxifen (Tam) at the age of either 6 or 9 weeks (FIG. 1). 100 μg/day of tamoxifen was injected for three consecutive days. Tamoxifen induces expression of Cre in keratinocytes resulting in deletion of the floxed p53.

Ptch1^{+/-}; K14-Cre-ER; p53^{fl/fl} mice and Ptch1^{+/-}; p53^{fl/fl} mice were irradiated at the age of 8 weeks by exposure to 5 Gray (Gy) of radiation (FIG. 1). Mice were observed for macroscopic tumor development.

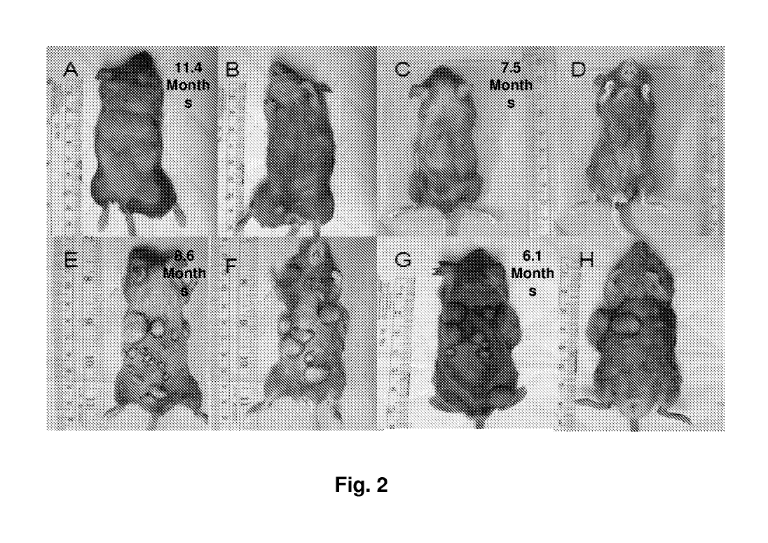

Deletion of p53 by injecting tamoxifen at the age of 6 weeks followed by irradiation at the age of 8 weeks resulted in an acceleration of BCC carcinogenesis in Ptch1^{+/-}; K14-Cre-ER; p53^{fl/fl} mice compared to Ptch1^{+/-}; p53^{fl/fl} mice (FIG. 2).

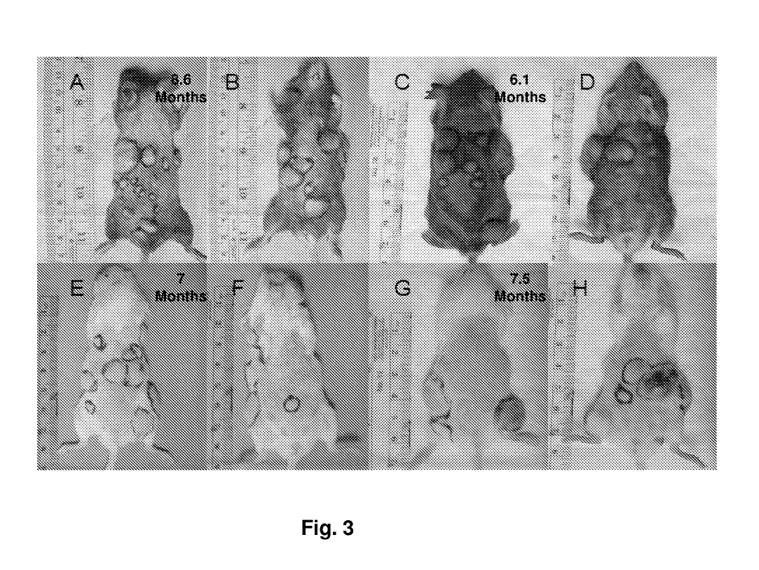

Deletion of p53 before and after irradiation accelerated BCC carcinogenesis. FIG. 3 shows mice of the genotype Ptch1^{+/-}; K14-Cre-ER; p53^{fl/fl}. Panels A-D show the mice that were injected with Tam at the age of 6 weeks and irradiated at 8 weeks of age (Exp1). Panels E-H show the mice that were irradiated at the age of 8 weeks and injected with Tam at the age of 9 weeks (Exp2).

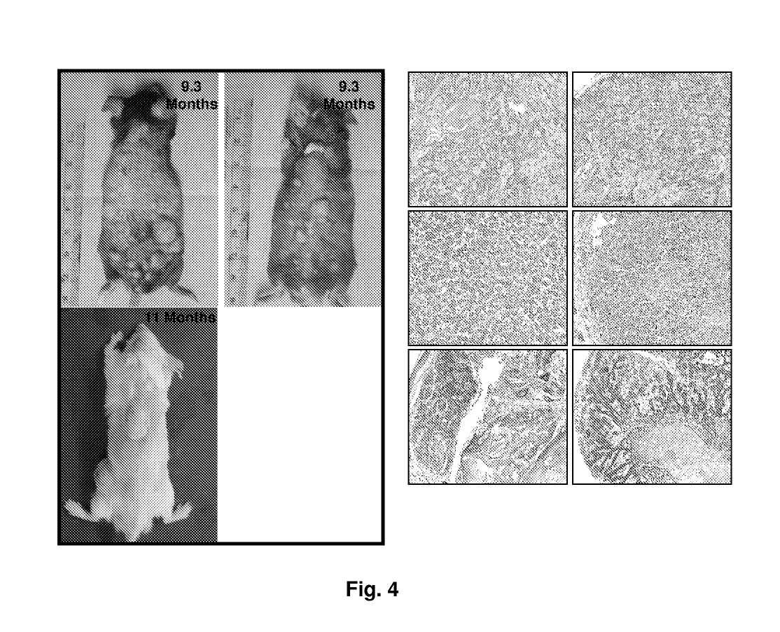

Deletion of p53 is sufficient to result in an acceleration of BCC carcinogenesis even in the absence of exposure to further mutagenesis by way of irradiation. FIG. 4 shows mice Ptch1^{+/-}; K14-Cre-ER; p53^{fl/fl} that were injected with Tam at the age of 6 weeks but were not irradiated (left panel) and histology of biopsied tumor from these mice (right panel).

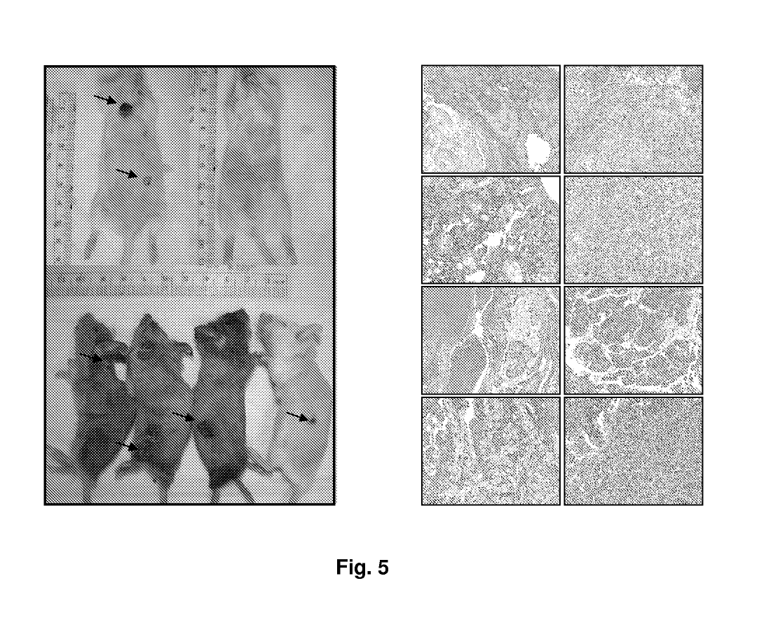

Mice of the genotype Ptch1^{+/+}; K14-Cre-ER; p53^{fl/fl} were generated to assess the phenotype associated with the deletion of p53 in keratinocytes while having the wild type Ptch1 gene. Ptch1^{+/+}; K14-Cre-ER; p53^{fl/fl} were generated by performing the following crosses: K14-Cre-ER transgenic mice were crossed with p53^{fl/fl} transgenic mice. Progenies from this cross having the genotype K14-Cre-ER; p53^{fl/+} were crossed to each other and progenies having the genotype K14-Cre-ER; p53^{fl/fl} were used for experiments. Mice of this genotype have the no mutation in the Ptch1 gene and hence are wild type (+/+) for Patch1 gene. Deletion of p53 by injecting Tam at the age of 6 weeks followed by irradiation at the age of 8 weeks causes the development of Squamous Cell Carcinoma (SCC) in mice that are wild type for Ptch1 gene (FIG. 5). Thus mice that lack expression of p53 protein in keratinocytes but have normal expression levels of Ptch1 protein develop SCC.

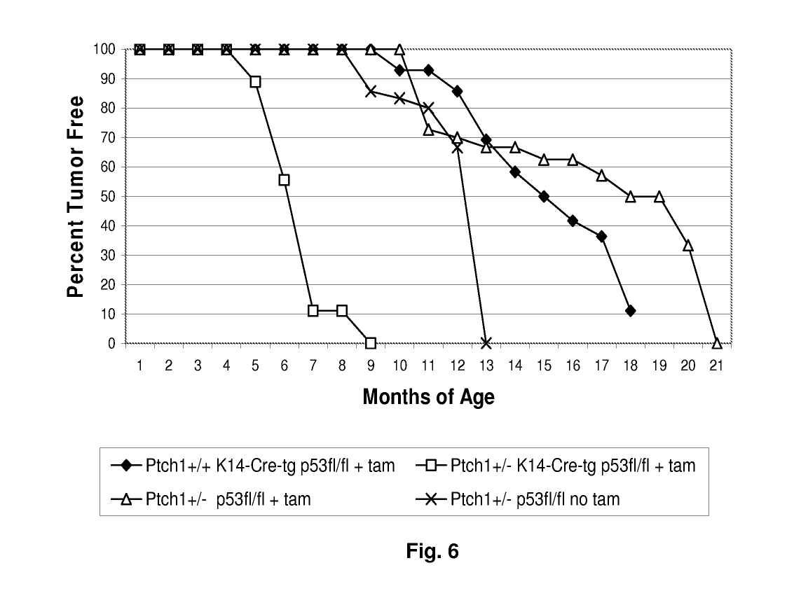

The percent of mice that were free of BCC was determined (FIG. 6). Mice of the genotype Ptch1^{+/-}; K14-Cre-ER; p53^{fl/fl} had a lower percent of mice that were free of BCC compared to mice of genotype Ptch1^{+/-}; p53^{fl/fl}. Mice were injected with Tam at the age of 6 weeks and irradiated at the age of 8 weeks. Ptch1^{+/-}; p53^{fl/fl} mice that were not injected with Tam served as an additional control group. Percent of mice of the genotype Ptch1^{+/+}; K14-Cre-ER; p53^{fl/fl} that were free of SCC when injected with Tam at the age of 6 weeks and irradiated at the age of 8 weeks was also determined (FIG. 6).

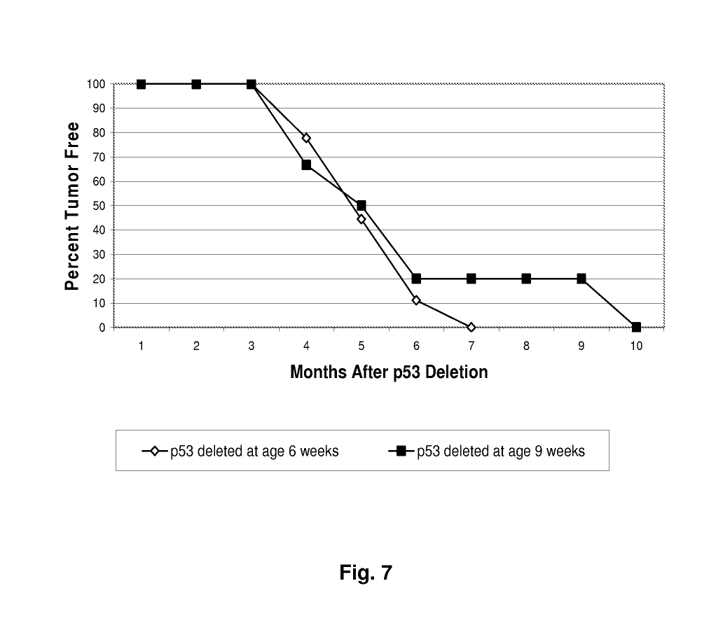

The effect of the timing of p53 deletion was determined by comparing the percent of mice that were tumor free (FIG. 7). Mice of the genotype Ptch1^{+/-}; K14-Cre-ER; p53^{fl/fl} that were injected with Tam at the age of 6 weeks and then irradiated at 8 weeks were compared to mice of the genotype Ptch1^{+/-}; K14-Cre-ER; p53^{fl/fl} that were irradiated at 8 weeks and then injected with Tam at the age of 9 weeks.

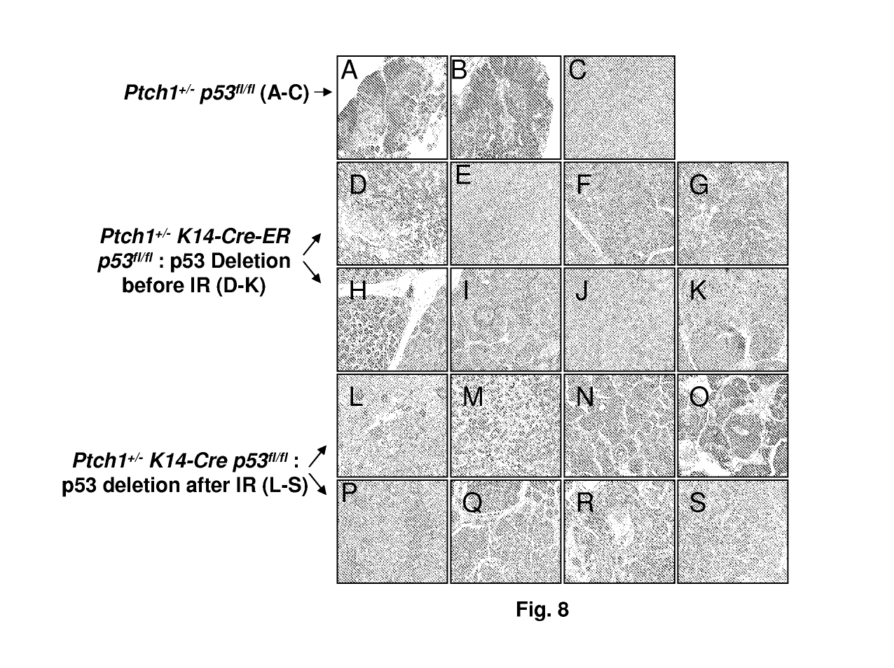

Histology of BCC biopsies was also examined (FIG. 8). Histology of BCC samples from Ptch1^{+/-}; p53^{fl/fl} were compared to that of Ptch1^{+/-}; K14-Cre-ER; p53^{fl/fl} that had deletion of p53 either before or after irradiation.

### Example 2

### Screening Candidate Agents

In order to screen candidate agents for anti-cancer activity, Ptch1^{+/-}; K14-Cre-ER; p53^{fl/fl} are injected with Tam at the age of 6 weeks and are irradiated at the age of 8 weeks with 5 Gray (Gy) of radiation. 100 μg/day of tamoxifen is injected for three consecutive days.

The candidate agents are administered after the mice develop BCC. These mice develop visible BCC at about 5 months of age. Mice having comparable BCC phenotype are divided into groups. One of the groups is administered a vehicle while the other groups are administered the candidate agents. The BCC phenotype is monitored at different time points, usually about once every month. The severity of BCC is determined based on the total number and size of BCC tumors. At different time points, BCC tumor is biopsied to examine its histology.

The average number of BCC per cm of the skin of mice in each group is determined Similarly, the average cross-sectional area of each BCC is measured for each group. A smaller average number of BCC or average cross-sectional area of BCC in mice administered a candidate agent compared to mice administered a vehicle indicates that the candidate agent has anti-cancer activity.

What is claimed is:

1. A method for screening anti-cancer agents comprising:
administering a candidate agent to a transgenic mouse having a heterozygous mutation in a Patched1 gene and having keratinocytes characterized by inducible suppression of functional p53 gene expression, wherein suppression of p53 gene expression is induced either prior to, with or after administration of said candidate agent;
wherein the presence or absence of an effect upon the cancer phenotype in said mouse is indicative of the anti-cancer activity of the candidate agent.

2. The method of claim 1, wherein the transgenic mouse is at least heterozygous for a floxed p53 gene and comprises at least one copy of a transgenic construct comprising a keratinocyte-specific promoter operably linked to a sequence encoding a fusion protein comprising Cre and at least a ligand-binding domain of an estrogen receptor (ER), wherein suppression of functional p53 gene expression in keratinocytes is induced by administration of a ligand for the ER ligand binding domain.

3. The method of claim 1, wherein the cancer is basal cell carcinoma.

4. The method of claim 1, wherein the transgenic mouse develops visible basal cell carcinoma within about 3 months of induction of suppression of functional p53 gene expression.

5. The method of claim 1, wherein said mouse is irradiated prior to, with or after administration of said candidate agent.

6. The method of claim 5, wherein said mouse is irradiated at the age of about 8 weeks.

7. A transgenic mouse comprising a heterozygous mutation in a Patched1 gene and having keratinocytes characterized by inducible suppression of functional p53 gene expression, wherein following suppression of functional p53 gene expression, the mouse develops basal cell carcinoma.

8. The transgenic mouse of claim 10, wherein the transgenic mouse is at least heterozygous for a floxed p53 gene and comprises at least one copy of a transgene construct comprising a keratinocyte-specific promoter operably linked to a sequence encoding a fusion protein comprising Cre and at least a ligand-binding domain of an estrogen receptor (ER), wherein suppression of functional p53 gene expression in keratinocytes is induced by administration of a ligand for the ER ligand binding domain.

9. The transgenic mouse of claim 8, wherein the mouse is homozygous for the floxed p53 gene.

10. The transgenic mouse of claim 7, wherein the transgenic mouse develops visible basal cell carcinoma within about 3 months after induction of suppression of functional p53 gene expression.
